(19) United States
(12) Patent Application Publication
(10) Pub. No.: US 2023/0365905 A1
(43) Pub. Date: Nov. 16, 2023

(54) COMBINED ALGAE PRODUCTION SYSTEM AND APPLICATION SYSTEM

(71) Applicant: SUN YAT-SEN UNIVERSITY, Guangzhou (CN)

(72) Inventors: Trevor Hocksun KWAN, Shenzhen (CN); Qinghe YAO, Shenzhen (CN); Yuxuan LUO, Shenzhen (CN)

(73) Assignee: SUN YAT-SEN UNIVERSITY, Guangzhou (CN)

(21) Appl. No.: 17/983,414

(22) Filed: Nov. 9, 2022

(30) Foreign Application Priority Data
May 11, 2022 (CN) 202210508606.0

(51) Int. Cl. C12M 1/00; C12M 1/02

(52) U.S. Cl. CPC C12M 21/02; C12M 23/22; C12M 41/18; C12M 47/18; C12M 47/02; C12M 29/18

(57) ABSTRACT

A combined algae bioreactor and heat-driven CO₂ capture system for algae production is provided. The combined system includes a bioreactor with a parabolic trough collector (PTC) shaped structure, a PTC top surface with a spectrum-splitting coating, a thermal solar receiver, a liquid inlet, a liquid outlet, a CO₂ feed pipeline, and gas release holes. The thermal solar receiver is arranged at the focal point of the bioreactor's PTC shape. The liquid inlet and the liquid outlet are arranged at two ends of a diagonal line of an opening of the bioreactor respectively. The CO₂ feed pipeline is connected to the bottom end of the bioreactor. The gas release holes are arranged at the two ends of the opening of the bioreactor. A spectrum-splitting coating is applied on the bioreactor's PTC top surface, which promotes algae production.

### CROSS REFERENCE TO THE RELATED APPLICATIONS

This application is based upon and claims priority to Chinese Patent Application No. 202210508606.0, filed on May 11, 2022, the entire contents of which are incorporated herein by reference.

### TECHNICAL FIELD

The present invention relates to the technical field of algae production, and in particular to a combined algae production system and application system.

### BACKGROUND

Algae is a highly effective energy carrier that can act as a biofuel or as food for animals and humans. Like common plants, algae cultivation consumes CO₂, which is a method for mitigating potential global warming. Moreover, typical algae species with practical value, including Chlorella vulgaris and Arthrospira platensis, generally grow and metabolize within one week, and consumes CO₂ faster than other common crops such as corn, rice, etc. Therefore, algae cultivation is the most suitable choice for achieving global warming mitigation.

A conventional algae bioreactor typically involves a transparent cylinder that contains an algae-water mixture and is placed directly under natural sunlight. A liquid flow loop and a separator are connected to the cylinder to circulate CO₂, O₂, produced algae, and other materials into and out of the bioreactor. Although such a reactor is simple in structure and cost-efficient, it cannot satisfy the standards required by mass production. Therefore, there is an urgent need to optimize the algae bioreactor design that maximizes yield, which is a major challenge because algae growth is highly sensitive to many environmental factors. Among them, providing moderately concentrated CO₂ (3% to 6%) can greatly enhance algae photosynthesis, and rapidly removing the by-product O₂ is needed to minimize the impact on algae cultivation. Secondly, illumination intensity shall be carefully regulated; A low illumination intensity will result in insufficient photosynthetic activity, and a high illumination intensity will result in a “photoinhibition” phenomenon, leading to excessive oxygen production by algae and inhibited activity. Finally, the reactor temperature should be in the range of 10° C. to 35° C. to guarantee the survival of the algae, and maximum growth is typically achieved at 30° C.

In the prior art, several reactor designs have been put forward that achieve better management of sunlight distribution, which enhances algae cultivation and provides a more uniform temperature distribution. These designs include long rectangular troughs, “race-way” like arrangements in long troughs, horizontal tubular reactors, and vertical tubular reactors. Furthermore, an alternative design also exists in which sunlight is concentrated into an optical fiber by applying a solar concentrator. Then, a set of light-diffusing optical rods are mounted on the other end of the optical fiber within the bioreactor, which redistributes the sunlight more uniformly than conventional algae bioreactor designs.

Nevertheless, the existing bioreactor designs have overlooked a major factor affecting the process's overall energy efficiency. Specifically, algae photosynthesis can only utilize light in the electromagnetic spectrum from 400 nm to 700 nm. Solar energy contains a very significant portion of energy outside this range, and these are typically transmitted, reflected, or converted into heat in the bioreactor. The conversion into heat is highly undesirable because it affects the temperature distribution of the bioreactor, which subsequently increases the difficulty to maintain optimal algae growth conditions. As a result, these unused parts of the solar spectrum lead to low solar energy efficiency and limits the maximum possible volume-specific yield rate of algae. Here, a common solution is to apply LEDs that only emit light in the desired spectrum of 400 nm to 700 nm as only needed by algae. However, LEDs consume electricity, which typically comes from photovoltaic panels in a spacecraft environment or coal power plants in terrestrial applications. The typical energy path of sunlight is: solar energy (or coal)→energy→LED lights→photosynthesis, which is extremely inefficient. Furthermore, the concentration of carbon dioxide in the atmosphere is about 400 ppm, which although sufficient for survival is very suboptimal for algae cultivation, and acquiring the desired carbon dioxide concentration (3% to 6%) from the atmospheric concentration of 400 ppm requires significant energy consumption. Although the exhaust gas of fossil fuel power plants (carbon dioxide concentration of roughly 10%) is a potential alternative solution, this approach is burdened by the harmful components such as SOₓ and NOₓ that exist in the flue gas and must be pretreated, and the excessive flue gas temperature (typically 150° C.) can destroy the algal environment altogether.

In summary, there is an urgent need to devise an alternative solution that increases the energy efficiency of algae bioreactors and provides the desired medium-concentration carbon dioxide without introducing other harmful constituents.

### SUMMARY

The present invention proposes a new technical scheme for solving the problems of low solar energy efficiency, insufficient CO₂ supply, and uneven temperature distributions as encountered in the prior art.

To achieve the above purpose, the present invention provides the following technical schemes.

A combined algae production system is provided, comprising: a bioreactor, a PTC top surface, a thermal solar receiver, a liquid inlet, a liquid outlet, a CO₂ feed pipeline, and gas release holes.

The bioreactor has a parabolic trough collector (PTC) shaped structure and an interior cavity.

The thermal solar receiver is positioned at the focal point of the bioreactor's PTC curve.

The liquid inlet and the liquid outlet are arranged at two ends of a diagonal line of an opening of the bioreactor respectively.

The CO₂ feed pipeline is connected to the bottom end of the bioreactor.

The gas release holes are arranged at the two ends of the opening of the bioreactor.

The bioreactor's opening surface is the PTC top surface.

The PTC top surface performs transmission and reflection of incident sunlight, where the spectral range that is favorable for algae photosynthesis is transmitted into the bioreactor space.

The other spectral ranges are reflected in the thermal solar receiver, which then performs thermal energy conversion.

The PTC top surface is a spectrum-splitting coating material.

Preferably, the thermal solar receiver has a cylindrical hollow structure.

The above-mentioned modified bioreactor design is then applied in a combined algae production system that further comprises a heat transfer fluid loop, an algae fluid loop, and a heat-driven CO₂ capture device.

The algae fluid loop is configured to circulate algae solution into and out of the new bioreactor design.

The heat transfer fluid loop is configured to transfer heat generated by the thermal solar receiver to the heat-driven CO₂ capture device that captures CO₂ from the atmosphere.

The captured CO₂ is then supplied to the combined algae production system.

Preferably, the heat transfer fluid loop comprises a liquid storage tank, two liquid pumps, and connecting pipelines.

One liquid pump provides liquid motion to allow the heat transfer fluid to collect heat from the thermal solar receiver via the pipelines.

The CO₂ capture device is connected to the liquid storage tank through a pipeline and a second liquid pump.

The second liquid pump provides a motion that allows the heat transfer fluid to supply collected heat to the heat-driven CO₂ capture device via the pipelines.

The liquid storage tank is used to stabilize the fluid temperature between the solar thermal receiver and the heat-driven CO₂ capture device and prevent abrupt temperature changes.

The CO₂ capture device receives the heat from a liquid storage tank and comprises a fan to receive air from the atmosphere. It conducts CO₂ capture on this air by consuming the supplied heat.

Preferably, the algae fluid loop comprises the bioreactor, an algae separator, and a third liquid pump that are sequentially connected in a series circuit.

The algae separator separates algae from water that originated from the bioreactor, and the third liquid pump provides motion to transfer the remaining solution back into the bioreactor.

Based on the above content, the present invention has the following benefits over the prior art:

The present invention promotes algae production by redirecting solar energy with electromagnetic spectrum ranges outside of 400 nm to 500 nm and 600 nm to 700 nm ranges (i.e., those that do not facilitate algae photosynthesis) to a heat-driven CO₂ capture device to capture CO₂ from atmospheric air. Thus, CO₂ of relatively higher concentration (e.g., greater than 1%) can be used for algae cultivation without the consumption of additional external energy. Since the present invention reflects the solar spectrum ranges that cannot be used for photosynthesis, this enables a more uniform temperature distribution and easier heat management in the bioreactor. The present invention is simple in design and only requires minor modifications to the geometry of a bioreactor. Otherwise, it could employ the same bioreactor wall material, liquid pumps, and algae separator that are already widely utilized by the prior art. Therefore, the present invention is cost-efficient and can be easily deployed at an industrial production scale.

### BRIEF DESCRIPTION OF THE DRAWINGS

To more clearly illustrate the technical schemes of the present invention, drawings are provided and are briefly introduced below. Indeed, these drawings are merely examples of the present invention and do not necessarily represent the complete scope of the present invention. Basic modifications such as choosing a different heat transfer fluid type, modifying the transmission and reflection spectral ranges of the spectral splitting film, etc. still involve applying the same concept as the present invention and thus are still considered as being within its scope.

FIG. 1 is a schematic that shows the modified algae bioreactor structure according to the present invention;

FIG. 2 is a block diagram of the combined algae production system that comprises the new algae bioreactor and according to the present invention; and

FIG. 3 is a graph that shows the ideal transmission and reflection spectral ranges by the spectrum splitting coating on the PTC top surface.

In the drawings: 1, bioreactor; 2, spectrum-splitting coating material; 3, thermal solar receiver; 4, incident sunlight; 5, reflected light; 6, transmitted light; 7, liquid inlet; 8, liquid outlet; 9, CO₂ feed pipeline; 10, gas release hole; 11, new bioreactor; 12, liquid storage tank; 13, CO₂ capture device, 14 & 15, liquid pump; 16, algae separator; 17, algae product; 18, atmospheric air; 19, captured CO₂.

### DETAILED DESCRIPTION OF THE EMBODIMENTS

The technical schemes in the embodiments of the present invention will be clearly described below and illustrated via the drawings. Based on these embodiments, all other embodiments that involve minor modifications but do not change the working principle of the present invention shall fall within this invention's protection scope.

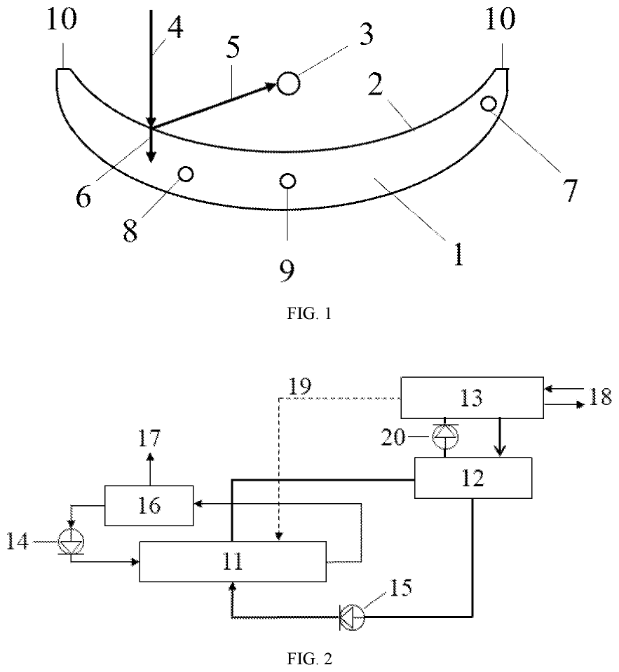

FIG. 1 discloses a modified bioreactor design that comprises a bioreactor 1, a PTC top surface, a thermal solar receiver 3, a liquid inlet 7, a liquid outlet 8, a CO₂ feed pipeline 9, and gas release holes 10.

Specifically, the bioreactor 1 has a parabolic trough collector (PTC) shaped structure and an interior cavity.

The thermal solar receiver 3 is positioned at the focal point of the bioreactor 1's PTC top surface.

The liquid inlet 7 and the liquid outlet 8 are arranged at two ends of a diagonal line at an opening of the bioreactor 1 respectively.

The CO₂ feed pipeline 9 is connected to the bottom of the bioreactor 1.

The gas release holes 10 are arranged at the two ends of the opening of the bioreactor 1.

The walls in bioreactor 1 are made of glass.

The PTC top surface comprises a glass wall that is coated with spectrum-splitting coating material 2, which is an ideal band-pass dichroic filter.

The thermal solar receiver 3 has a cylindrical hollow structure.

The material of the thermal solar receiver 3 can be graphite and ideally should have an absorption coefficient of 1 at all electromagnetic wavelengths to photothermally convert the reflected light 5 into heat.

During operation, incident sunlight 4 is incident on the PTC top surface of bioreactor 1, which contains light within the broad range of the electromagnetic spectrum (approx. 200 nm to 2500 nm).

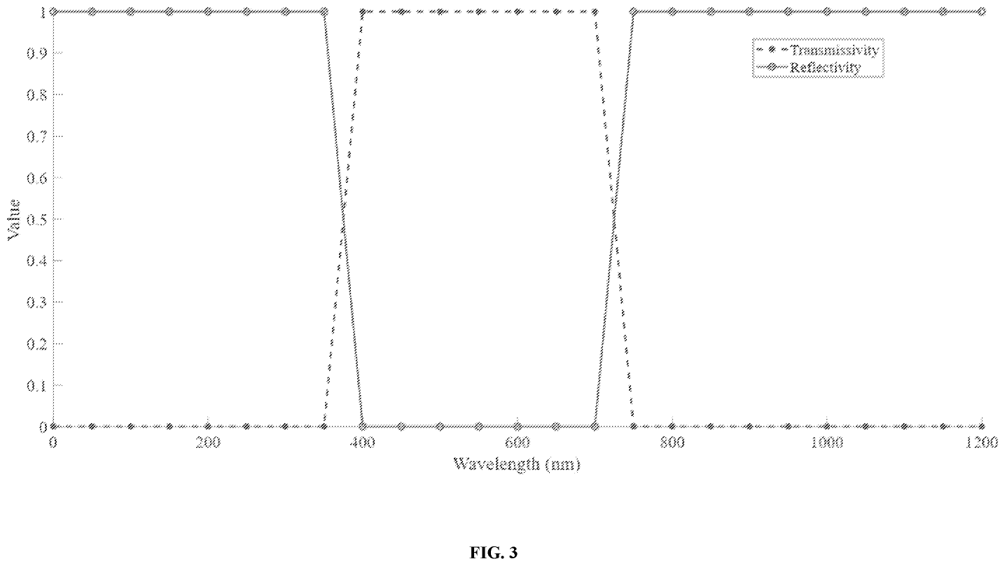

The PTC top surface performs transmission and reflection of incident sunlight, where the spectral range that is favorable for algae photosynthesis (400 nm to 500 nm and 600 nm to 700 nm) is transmitted into the bioreactor space. Specifically, the transmitted light is marked as 6 in FIG. 3 and is progressively absorbed by the algae as it travels through the bioreactor space;

Meanwhile, the other spectral ranges (marked as reflected light 5 in FIG. 3) are reflected towards the thermal solar receiver, which then performs thermal energy conversion.

FIG. 2 discloses the combined algae production system that comprises the modified bioreactor design 11 of FIG. 1, a heat transfer fluid loop, an algae fluid loop, and a heat-driven CO2 capture device.

The algae fluid loop circulates the algae solution into and out of the modified bioreactor design 11.

The heat transfer fluid loop is configured to transfer heat generated by the modified bioreactor design 11 to a CO₂ capture device 13 to capture CO₂, and the captured CO₂ is passed into the bioreactor 1.

The heat transfer fluid loop comprises a liquid storage tank 12, and two liquid pumps 15 and 20.

The first liquid pump 15, the thermal solar receiver 3, and the liquid storage tank are sequentially connected in a series circuit.

The first liquid pump 15 pumps fluid through the solar thermal receiver 3 to collect and transfer the photothermally generated heat into the liquid storage tank.

The second liquid pump 20, the heat-driven CO₂ capture device 13, and the liquid storage tank are sequentially connected in a series circuit.

The liquid storage tank's purpose is to maintain a relatively consistent temperature for the heat-driven CO₂ capture device regardless of fluctuations and intermittent inputs from the thermal solar receiver 3. The liquid storage tank may be any conventional corrosion-resistant tank and should be sized according to the maximum tolerable temperature fluctuation requirement of the heat-driven CO₂ capture device.

The heat-driven CO₂ capture device 13 may be any method that consumes heat to capture CO₂, which may include chemical absorption or physical adsorption. In the below description, an example is described by considering a typical adsorption technology that operates in a cyclic manner between the adsorption and regeneration modes. During adsorption, atmospheric air is continuously blown through an adsorbent material to adsorb CO₂ from the flowing air. Then, during regeneration, the adsorbent material is isolated from the atmosphere, and high-temperature heat should be applied to regenerate the material (i.e., release the adsorbed CO₂ and make it gaseous). Here, a fluid heat exchanger is installed within the heat-driven CO₂ capture device, and the second liquid pump 20 pumps fluid from the liquid storage tank to this fluid exchanger to provide the required heat for the regeneration process.

The algae fluid loop comprises the bioreactor 1, an algae separator 16, and the third liquid pump 14 that are sequentially connected in a series circuit.

The algae separator 16 separates algae from the water solution that originated from the bioreactor 1, and the third liquid pump 14's pumping action allows the remaining solution to return to the bioreactor 1.

The fluid used in the heat transfer fluid loop can be any common non-toxic liquid provided that it remains as a liquid at the operating temperatures of the thermal solar receiver 3 (≈100° C.). A typical example is a water-glycol solution.

The algae separator 16 can be comprised of any type of conventional separation technology. In the present description, a separation method based on a 3-way valve is described. Its operation is as follows. The three-way valve is first opened to allow an outflow of the algae-water solution to a collection tank. Then, this collected algae-water solution is further treated in a separation machine such as a membrane separator, a precipitator, flocculation, etc. After a sufficient amount of the algae-water solution is removed, the 3-way valve is then switched to allow the entrance of clean water to replenish that lost from the previously removed algae-water solution.

Overall, in the present invention, the concept of redirecting the electromagnetic spectrums that are outside the ideal range for photosynthesis has brought many beneficial features that ultimately increased the specific volume of algae growth rate. Firstly, by reflecting the unusable spectrums for photosynthesis, the potential for large heat generation within the bioreactor space is avoided. This is beneficial especially when the algae bioreactor 1 operates in a humid hot climate (e.g. >33° C., RH>75%) where a slight increase in temperature could significantly lower the algae growth rate. Secondly, the unusable spectrums are, for the first time, effectively utilized as an energy source for the capture of CO₂ from the atmosphere. Thus, CO₂ that is void of harmful contaminants (e.g., NOx, SOx, etc.) with concentrations optimal for algae growth (3% to 6%) can be used for algae cultivation without the consumption of significant external energy. In summary, the present invention is simple in design and only requires minor modifications to the geometry of a bioreactor 1. The other components in the present invention, such as an algae water loop and the bioreactor 1 are basic components that would already be required by a conventional bioreactor system, and the HTF fluid loop is easily constructible from commercial parts. Therefore, the present invention is economically feasible and can be easily deployed at industrial scales.

What is claimed is:

1. A combined algae production system, comprising: a bioreactor, a parabolic trough collector (PTC) top surface, a thermal solar receiver, a liquid inlet, a liquid outlet, a CO₂ feed pipeline, and gas release holes, wherein
the bioreactor has a parabolic trough collector-shaped structure and an interior cavity structure;
the thermal solar receiver is arranged at a focal point of the bioreactor;
the liquid inlet and the liquid outlet are arranged at two ends of a diagonal line of an opening of the bioreactor respectively;
the CO₂ feed pipeline is connected to a bottom end of the bioreactor;
the gas release holes are arranged at the two ends of the opening of the bioreactor; and
an opening surface of the bioreactor is the PTC top surface.

2. The combined algae production system according to claim 1, wherein the PTC top surface performs transmission and reflection of a spectrum of incident sunlight, transmits a first part of the spectrum to the bioreactor, and reflects a second part of the spectrum to the thermal solar receiver; and
the thermal solar receiver receives the second part of the spectrum reflected by the PTC top surface and performs thermal conversion.

3. The combined algae production system according to claim 1, wherein the PTC top surface is a spectrum-splitting coating material.

4. The combined algae production system according to claim 1, wherein the thermal solar receiver has a cylindrical hollow structure; and
a position of the thermal solar receiver matches with the focal point of the bioreactor's PTC surface.

5. A combined algae production application system, comprising:
the combined algae production system according to claim 1,
a heat transfer fluid loop, and
an algae fluid loop;
wherein the algae fluid loop is configured to circulate algae solution into and out of the combined algae production system; and
the heat transfer fluid loop is configured to transfer heat generated by the combined algae production system to a CO₂ capture device to capture CO₂, and convey the CO₂ to the combined algae production system.

6. The combined algae production application system according to claim 5, wherein the heat transfer fluid loop comprises: a liquid storage subsystem and a CO₂ capture subsystem; wherein
the liquid storage subsystem supplies the heat generated by the combined algae production system to the CO₂ capture subsystem through a first pipeline.

7. The combined algae production application system according to claim 6, wherein the liquid storage subsystem comprises: a liquid storage tank, a second liquid pump, and the thermal solar receiver, wherein the liquid storage tank, the second liquid pump and the thermal solar receiver are sequentially connected in a series circuit; and
the liquid storage tank stores heat generated by the thermal solar receiver, and triggers flow in the heat transfer fluid loop through the second liquid pump.

8. The combined algae production application system according to claim 6, wherein the CO₂ capture subsystem comprises: the CO₂ capture device and a liquid storage tank;
the CO₂ capture device is connected to the liquid storage tank through a second pipeline;
the CO₂ capture device receives heat and atmospheric air stored in the liquid storage tank, and achieves CO₂ capture by consuming the heat.

9. The combined algae production application system according to claim 5, wherein
the algae fluid loop comprises: the bioreactor, an algae separator, and a first liquid pump, wherein the bioreactor, the algae separator and the first liquid pump are sequentially connected in a series circuit;
the algae separator separates algae and water produced by the bioreactor, and triggers flow in the algae fluid loop through the second liquid pump to transfer remaining water back to the bioreactor.

10. The combined algae production application system according to claim 5, wherein the PTC top surface performs transmission and reflection of a spectrum of incident sunlight, transmits a first part of the spectrum to the bioreactor, and reflects a second part of the spectrum to the thermal solar receiver; and
the thermal solar receiver receives the second part of the spectrum reflected by the PTC top surface and performs thermal conversion.

11. The combined algae production application system according to claim 5, wherein the PTC top surface is a spectrum-splitting coating material.

12. The combined algae production application system according to claim 5, wherein the thermal solar receiver has a cylindrical hollow structure; and
a position of the thermal solar receiver matches with the focal point of the bioreactor's PTC surface.
